(19) United States
(12) Patent Application Publication
(10) Pub. No.: US 2024/0374637 A1
(43) Pub. Date: Nov. 14, 2024

(54) COMPOSITIONS AND METHODS FOR ENHANCING STEM CELL SURVIVAL

(71) Applicant: The Regents of the University of California, Oakland, CA (US)

(72) Inventor: Anahid Jewett, Los Angeles, CA (US)

(21) Appl. No.: 18/285,442

(22) PCT Filed: Apr. 5, 2022
(86) PCT No.: PCT/US22/23398

§ 371 Date: Oct. 3, 2023

Related U.S. Application Data
(60) Provisional application No. 63/171,189, filed on Apr. 6, 2021.

(51) Int. Cl. A61K 35/15; A61K 35/28; A61K 39/00; A61K 45/06; C12N 5/0775; C12N 5/0786

(52) U.S. Cl. CPC A61K 35/15; A61K 35/28; A61K 39/4614; A61K 39/464429; A61K 45/06; C12N 5/0645; C12N 5/0663; A61K 2239/47; C12N 2500/02; C12N 2529/00

(57) ABSTRACT

The present invention relates to compositions that comprise monocytes that enhances the survival of stem cells and methods of transplanting such compositions for treating various conditions.

### CROSS-REFERENCE TO RELATED APPLICATIONS

This application claims the benefit of U.S. Provisional Application No. 63/171,189, filed on Apr. 6, 2021, the entire contents of which are incorporated herein in their entirety by this reference.

### BACKGROUND

Stem cells are the body's raw materials—cells from which all other cells with specialized functions are generated. Stem cell transplantation allows the recipient to replace damaged cells, thereby preventing or treating a wide range of conditions/diseases such as neurodegenerative diseases, cancer, ischemic heart disease, and paralysis.

Notably, neurodegenerative disorders are incurable and debilitating conditions that result in progressive degeneration and/or death of nerve cells. Amyotrophic Lateral Sclerosis (ALS) is the most common adult-onset motor neuron disease or neuromuscular disorder, caused by the progressive degeneration of motor neurons in the spinal cord, brainstem, and motor cortex, resulting in loss of muscle control (Rowland et al., (2005) N Engl J Med 344:1688-1700). Motor neurons reach from the brain to the spinal cord and from the spinal cord to the muscles throughout the body. The progressive degeneration of the motor neurons in ALS eventually leads to death. When the motor neurons die, the ability of the brain to initiate and control muscle movement is lost, and patients in the later stages of the disease often become paralyzed.

While effective, stem cell transplantation is challenging, largely due to a low survival rate of the transplanted stem cells. It has been reported that only 6% of the stem cells injected into an animal model of ischemic myocardium survive at 10 days after injection. In human, the survival of the transplanted stem cells range from 2-6% when measured 2 days after injection (Li et al. (2016) Stem Cells Int. 9682757:1-14).

Accordingly, there is a great need for compositions and methods for enhancing the survival of stem cells.

### SUMMARY OF THE INVENTION

The present invention is based, at least in part, on the discovery that monocytes (e.g., CD16+ monocytes) are particularly useful in enhancing the survival of stem cells (e.g., mesenchymal stem cells (MSCs), embryonic stem cells (ESCs), dental pulp stem cells (DPSCs), hematopoietic stem cells (HSCs), and induced pluripotent stem cell (iPSCs)). Accordingly, compositions and methods of the present disclosure are effective in preventing and treating various conditions/diseases such as a neurodegenerative disease (e.g., ALS), paralysis, cancer (e.g., leukemia, lymphoma, multiple myeloma), liver cirrhosis, or ischemic heart disease.

In certain aspects, provided herein is a pharmaceutical composition comprising irradiated monocytes and stem cells.

In certain embodiments, the stem cells are preconditioned to enhance the survival. For example, the stem cells may be preconditioned with exposure to hypoxia (e.g., 0.5% oxygen), hyperoxia (100% oxygen), and/or sevoflurane.

In certain embodiments, the pharmaceutical composition of the present disclosure further comprises at least one additional agent that enhances the survival of the stem cells. In some embodiments, the at least one additional agent is selected from TGF-α, Z-VAD-FMK pan-caspase inhibitor, simvastatin, rosuvastatin, an inhibitor of Janus kinase (JAK), and an inhibitor of p38.

In certain embodiments, the pharmaceutical composition comprises a biomaterial, optionally wherein the biomaterial comprises hydrogel and/or a cell sheet.

In certain embodiments, the stem cells are selected from mesenchymal stem cells (MSCs), embryonic stem cells (ESCs), dental pulp stem cells (DPSCs), hematopoietic stem cells (HSCs), stem cells from human exfoliated deciduous teeth (SHED), and induced pluripotent stem cell (iPSCs).

In certain embodiments, the monocytes are gamma-irradiated.

In certain embodiments, the composition comprises monocytes that express CD16 on the cell surface. For example, in some embodiments, at least 50%, 60%, 70%, 80%, 90%, or 95% of the monocytes in the composition express CD16 on the cell surface.

In some embodiments, the composition produces or secretes at least one cytokine or growth factor. For example, in some embodiments, the at least one cytokine or growth factor is selected from IL-6, TNF-α, and VEGF. In some embodiments, the at least one cytokine or growth factor is IL-6, TNF-α, and VEGF.

In some embodiments, the monocytes increases the activity of NFkB in the stem cells.

In some embodiments, the monocytes (i) inhibit the NK cell-mediated death of the stem cells, and/or (ii) enhance the survival of the stem cells.

In certain aspects, also provided herein is a method of treating a subject in need of stem cell transplantation, the method comprising transplanting the composition of the present disclosure.

For example, in certain embodiments, the subject is afflicted with a neurodegenerative disease, paralysis, cancer (e.g., leukemia, lymphoma, multiple myeloma), liver cirrhosis, or ischemic heart disease.

In certain embodiments, a peripheral nerve function is decreased in the subject.

In some embodiments, the subject is afflicted with a traumatic nerve injury, muscle atrophy, muscular denervation atrophy, neuropathy, or motor neuron disease.

In some embodiments, the subject is afflicted with a peripheral nerve injury, amyotrophic lateral sclerosis (ALS), spinal muscular atrophy, spinocerebellar ataxia, post-polio syndrome, and Charcot-Marie-Tooth disease.

In certain embodiments, a central nerve function is decreased in the subject.

In some embodiments, the subject is afflicted with Alzheimer's disease, Parkinson's disease, or Huntington's disease.

In certain aspects, further provided herein is a method of promoting neuromuscular regeneration in a subject, the method comprising transplanting the composition of the present disclosure.

In some embodiments, the subject is afflicted with a neurodegenerative disease.

In some embodiments, a peripheral nerve function is decreased in the subject.

In some embodiments, the subject is afflicted with a traumatic nerve injury, muscle atrophy, muscular denervation atrophy, neuropathy, or motor neuron disease.

In some embodiments, the subject is afflicted with a peripheral nerve injury, amyotrophic lateral sclerosis (ALS), spinal muscular atrophy, post-polio syndrome, spinocerebellar ataxia, or Charcot-Marie-Tooth disease.

In certain embodiments, a central nerve function is decreased in the subject.

In some embodiments, the subject is afflicted with Alzheimer's disease, Parkinson's disease, or Huntington's disease.

Numerous embodiments are further provided that can be applied to any aspect of the present invention and/or combined with any other embodiment described herein. For example, in certain embodiments, the monocytes are autologous or allogeneic to the subject.

In certain embodiments, the stem cells are autologous or allogeneic to the subject.

In certain embodiments, the methods of the present disclosure further comprise administering to the subject at least one additional agent that enhances the survival of the stem cells. In some such embodiments, the at least one additional agent is administered before, after, or concurrently with the composition of the present disclosure (e.g., those comprising the cells described herein). In some embodiments, the at least one additional agent is selected from TGF-α, Z-VAD-FMK pan-caspase inhibitor, simvastatin, rosuvastatin, an inhibitor of Janus kinase (JAK), and an inhibitor of p38.

In certain embodiments, the composition is transplanted intramuscularly or by intraosseus infusion.

In some embodiments, the subject is a mammal. In preferred embodiments, the subject is a human.

In certain aspects, further provided herein is a method of enhancing the survival of stem cells, the method comprising contacting the stem cells with monocytes, e.g., in vitro, ex vivo, or in vivo.

In certain embodiments, the monocytes express CD16 on the cell surface. For example, in some embodiments, at least 50%, 60%, 70%, 80%, 90%, or 95% of the monocytes, which contact the stem cells, express CD16 on the cell surface.

In certain embodiments, the monocytes are irradiated, e.g., gamma irradiated.

In certain embodiments, the stem cells are selected from mesenchymal stem cells (MSCs), embryonic stem cells (ESCs), dental pulp stem cells (DPSCs), hematopoietic stem cells (HSCs), stem cells from human exfoliated deciduous teeth (SHED), and induced pluripotent stem cell (iPSCs).

In certain embodiments, the monocytes produce or secrete at least one cytokine or growth factor in the presence of the stem cells. For example, in some embodiments, the at least one cytokine or growth factor is selected from IL-6, TNF-α, and VEGF. In some embodiments, the at least one cytokine or growth factor comprises IL-6, TNF-α, and VEGF.

In preferred embodiments, the monocytes increase the activity of NFkB in the stem cells.

In some embodiments, the monocytes (i) inhibit the NK cell-mediated death of the stem cells, and/or (ii) enhance the survival of the stem cells.

In certain embodiments, the methods of the present disclosure further comprise contacting the stem cells with at least one additional agent that enhances the survival of the stem cells. In some embodiments, the at least one additional agent is selected from TGF-α, Z-VAD-FMK pan-caspase inhibitor, simvastatin, rosuvastatin, an inhibitor of Janus kinase (JAK), and an inhibitor of p38.

In certain embodiments, the stem cells are preconditioned to enhance their survival, e.g., by exposure to hypoxia (e.g., 0.5% oxygen), hyperoxia (100% oxygen), or sevoflurane.

### BRIEF DESCRIPTION OF FIGURES

FIG. 1. Potent lysis of MSCs by the Natural Killer cells: Inhibition by anti-CD16 antibodies. Highly purified NK cells were left untreated or treated with anti-CD16 mAb (3 μg/ml), IL-2 (1000 u/ml), or a combination of IL-2 and anti-CD16 mAb for 8-12 hours before they were added to the ⁵¹Cr labeled MSCs (FIG. 1A) or ⁵¹Cr labeled K562 (FIG. 1B) at different effector to target (E:T) ratios. After 4 hours of incubation the supernatants were removed and the released radioactivity counted by a γ counter. % cytotoxicity was determined as indicated in the Materials and Methods section. One of five representative experiments is shown in this figure.

FIG. 2. Monocytes protect MSCs against NK cell mediated cytotoxicity. Autologous monocytes were purified from PBMCs and irradiated as indicated in the Material and Methods section. MSCs were co-cultured with irradiated monocytes (monocyte:MSC ratio of 1:1) for 24-48 hours before they were labeled with ⁵¹Cr and used as targets in the cytotoxicity assays against NK cells. The NK samples were either left untreated (FIG. 2A) or treated with anti-CD16 mAb (3 μg/ml) (FIG. 2B), IL-2 (1000 u/ml) (FIG. 2C), or a combination of IL-2 and anti-CD16 mAb (FIG. 2D) for 24-48 hours before they were added to ⁵¹Cr labeled MSCs at different effector to target (E:T) ratios. Supernatants were removed after 4 hours of incubation and the released radioactivity counted by a γ counter. % cytotoxicity was determined as indicated in the Material and Methods section. One of three representative experiments is shown in this figure.

FIG. 3. Monocytes trigger secretion of IL-6, TNF-α, and VEGF by MSCs. MSCs were co-cultured with and without irradiated Monocytes at 1:1 MSCs to Monocytes for 24-48 hours before supernatants were removed and the levels of IL-6 (FIG. 3A), TNF-α (FIG. 3B), VEGF (FIG. 3C), were determined using multiplex cytokine array kit. The results were also confirmed for each cytokine measured using single ELISAs. One of three representative experiments is shown in this figure.

FIG. 4. Monocytes are potent inducers of NFkB in HEp2 cells. HEp2 cells were transfected with NFkB luciferase reporter vector before they were co-cultured with Monocytes, purified NK cells, Peripheral Blood Lymphocytes (PBLs), Polymorphonuclear (PMNs) and Peripheral Blood Mononuclear Cells (PBMCs) at 1:1 ratio. After 4 hours of incubation the fold induction of NFkB activity in the samples were determined over the control HEp2 cells in the absence of immune effectors (FIG. 4A). HEp2 cells were transfected with NFkB luciferase reporter vector before they were co-cultured with Total monocytes and CD16− subsets of Monocytes, purified untreated and IL-2 (1000 u/ml) treated NK cells, and untreated and IL-2 (1000 u/ml) treated Peripheral Blood Lymphocytes at 1:1 ratio. After 4 hours of incubation the fold induction of NFkB activity was determined over the control HEp2 cells in the absence of immune effectors (FIG. 4B). One of eight representative experiments is shown in this figure.

FIG. 5. Total Monocytes and CD16− subsets of Monocytes protect MSCs against NK cell mediated cytotoxicity. MSCs were co-cultured with highly purified and irradiated total monocytes and CD16− subsets of Monocytes at 1:1 MSCs to Monocytes for 24-48 hours before MSCs were labeled with 51Cr and added to untreated or IL-2 (1000 u/ml) pretreated or anti-CD16mAb (3 μg/ml) pre-treated, or a combination of IL-2 and anti-CD16 mAb pre-treated NK cells. NK cells were pre-treated as indicated for 24-48 hours before they were added to the co-cultures of monocytes and MSCs. After 4 hours of incubation the supernatants were removed and the released radioactivity counted by a γ counter. % cytotoxicity was determined, and LU₃₀/10⁷ cells were calculated using the inverse of the number of effectors needed to lyse 30% of the MSCsX100 (FIG. 5).

### DETAILED DESCRIPTION OF THE INVENTION

The present invention is based, at least in part, on the discovery that monocytes (e.g., CD16+ monocytes) are particularly useful in enhancing the survival of stem cells (e.g., mesenchymal stem cells (MSCs), embryonic stem cells (ESCs), dental pulp stem cells (DPSCs), hematopoietic stem cells (HSCs), and induced pluripotent stem cell (iPSCs)). Accordingly, compositions and methods of the present disclosure are effective in preventing and treating various conditions/diseases such as a neurodegenerative disease (e.g., ALS), paralysis, cancer (e.g., leukemia, lymphoma, multiple myeloma), liver cirrhosis, or ischemic heart disease.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow a therapy to perform its intended function. Examples of routes of administration include injection (intramuscular, subcutaneous, intravenous, parenterally, intraperitoneally, intrathecal, intraosseous infusion (i.e., the process of injecting directly into the marrow of a bone), etc.) routes. The injection can be a bolus injection or can be a continuous infusion. Depending on the route of administration, the agent can be coated with or disposed in a selected material to protect it from natural conditions which may detrimentally affect its ability to perform its intended function.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the products in the test sample. Such a control may comprise any suitable sample, including but not limited to a sample from a control diseased patient (e.g., those afflicted with e.g., a neurodegenerative disease, paralysis, cancer (e.g., leukemia, lymphoma, multiple myeloma) (can be stored sample or previous sample measurement) with a known outcome; normal tissue or cells isolated from a subject, such as a normal patient or the diseased patient (e.g., those afflicted with an inflammatory/autoimmune disease or cancer), cultured primary cells/tissues isolated from a subject such as a normal subject or the diseased patient, adjacent normal cells/tissues obtained from the same organ or body location of the diseased patient, a tissue or cell sample isolated from a normal subject, or a primary cells/tissues obtained from a depository. In other embodiments, the control may comprise a reference standard (e.g., a standard cytokine/chemokine level) from any suitable source, including but not limited to a previously determined cytokine/chemokine level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard cytokine/chemokine levels can be used in combination as controls in the methods of the present invention. In some embodiments, the control may comprise normal or non-diseased cell/tissue sample. In other embodiments, the control may comprise a level for a set of patients, such as a set of diseased patients, or for a set of patients receiving a certain treatment, or for a set of patients with one outcome versus another outcome. In other embodiments, the control may comprise normal cells, cells from patients treated with combination chemotherapy, and cells from patients having benign cancer. In still other embodiments, the control may also comprise a measured value for example, healthy or diseased individuals who were not treated with the compositions of the present disclosure, or healthy or diseased individuals who were treated with other therapies. In other embodiments, the control comprises a ratio, e.g., of cytokine/chemokine levels, including but not limited to the level of one cytokine against the level of another cytokine. In particularly preferred embodiments, the control comprises a control sample which is of the same lineage and/or type as the test sample. Such “control” is not limited for comparing the level of cytokine/chemokine but can also be applied to any other parameter described herein, such as survival of the stem cells, expression, inhibition, cytotoxicity, cell growth, and the like.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state.

A “kit” is any manufacture (e.g. a package or container) comprising at least one reagent or a composition (e.g., a pharmaceutical composition) of the present disclosure (e.g., monocytes (e.g., irradiated and/or CD16+) with or without stem cells). The kit may be promoted, distributed, or sold as a unit for performing the methods of the present invention. The kit may comprise one or more reagents necessary to produce a composition useful in the methods of the present invention. In certain embodiments, the kit may also include instructional materials disclosing or describing the use of the kit. A kit may also include additional components to facilitate the particular application for which the kit is designed.

The term “preventing” is art-recognized, and when used in relation to a condition, such as a local recurrence (e.g., loss of muscle control), a disease such as neurodegenerative disorder (e.g., ALS), a syndrome complex such as paralysis or any other medical condition, is well understood in the art, and includes administration of a composition which reduces the frequency of, or delays the onset of, symptoms of a medical condition in a subject relative to a subject which does not receive the composition.

The amount or activity of e.g., cytokines or chemokines is “significantly” higher or lower than the normal amount of the cytokines or chemokines, if the amount is greater or less, respectively, than the normal level by an amount greater than the standard error of the assay employed to assess amount, and preferably at least, about, or no more than 10%, 20%, 30%, 40%, 50%, 60%, 70%, 80%, 90%, 100%, 150%, 200%, 300%, 350%, 400%, 500%, 600%, 700%, 800%, 900%, 1000% or more than that amount. Alternately, the amount of the cytokines and/or chemokines can be considered “significantly” higher or lower than the normal amount if the amount is at least about two, and preferably at least about three, four, or five times, higher or lower, respectively, than the normal amount of the cytokines or chemokines. Such “significance” can also be applied to any other measured parameter described herein, such as for survival of the stem cells, expression, inhibition, cytotoxicity, cell growth, and the like.

The term “subject” or “patient” refers to any healthy or diseased animal, mammal or human, or any animal, mammal or human. In some embodiments, the subject is afflicted with a neurodegenerative disease, paralysis, cancer (e.g., leukemia, lymphoma, multiple myeloma), liver cirrhosis, or ischemic heart disease. In various embodiments of the methods of the present invention, the subject has not undergone treatment. In other embodiments, the subject has undergone treatment.

A “therapeutically effective amount” of a substance or cells is an amount capable of producing a medically desirable result in a treated patient, e.g., increase muscle control, delay or reduce paralysis, or alleviate any symptom associated with a disease (e.g., a neurodegenerative disease, paralysis, cancer (e.g., leukemia, lymphoma, multiple myeloma), liver cirrhosis, or ischemic heart disease) with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Cytokines, Chemokines, Growth Factors

Cytokines are small, non-structural proteins of inflammation and immunology. Cytokines affect nearly every biological process; these include embryonic development, disease pathogenesis, non-specific response to infection, specific response to antigen, changes in cognitive functions and progression of the degenerative processes of aging. In addition, cytokines are part of stem cell differentiation, vaccine efficacy and allograft rejection. Multiple biological properties or pleiotropism is the hallmark of a cytokine, and cytokines encompass interferons, the interleukins, chemokines, lymphokines, mesenchymal growth factors, the tumor necrosis factor family and adipokines.

An inflammatory cytokine or proinflammatory cytokine is a type of signaling molecule (a cytokine) that is secreted from immune cells, e.g., helper T cells (Th) and macrophages, and certain other cell types that promote inflammation. They include interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions.

Excessive chronic production of inflammatory cytokines contribute to inflammatory diseases, that have been linked to different diseases, such as atherosclerosis and cancer. Dysregulation has also been linked to depression and other neurological diseases. A balance between proinflammatory and anti-inflammatory cytokines is necessary to maintain health. Aging and exercise also play a role in the amount of inflammation from the release of proinflammatory cytokines.

The major proinflammatory cytokines that are responsible for early responses are IL-1-alpha, IL-1-beta, IL-6, and TNF-α. Other proinflammatory mediators include members of the IL-20 family, IL-33 LIF, IFN-gamma, OSM, CNTF, TGF-beta, GM-CSF, IL-11, IL-12, IL-17, IL-18, IL-8, Rantes, and a variety of other chemokines that chemoattract inflammator cells. These cytokines either act as endogenous pyrogens (IL-1, IL-6, TNF-α), upregulate the synthesis of secondary mediators and proinflammatory cytokines by both macrophages and mesenchymal cells (including fibroblasts, epithelial and endothelial cells), stimulate the production of acute phase proteins, or attract inflammatory cells.

IL-6 has been shown to play a central role in the neuronal reaction to nerve injury. Suppression of IL-6R by in vivo application of anti-IL-6R antibodies led to reduced regenerative effects. IL-6 is also involved in microglial and astrocytic activation as well as in regulation of neuronal neuropeptides expression. There is evidence that IL-6 contributes to the development of neuropathic pain behavior following a peripheral nerve injury. For example, sciatic cryoneurolysis, a sympathetically-independent model of neuropathic pain involving repeatedly freezing and thawing a section of the sciatic nerve, results in increased IL-6 immunoreactivity in the spinal cord. In addition, intrathecal infusion of IL-6 induces tactile allodynia and thermal hyperalgesia in intact and nerve-injured rats, respectively.

TNF-α, also known as cachectin, is another inflammatory cytokine that plays a well-established, key role in some pain models. TNF acts on several different signaling pathways through two cell surface receptors, TNFR1 and TNFR2 to regulate apoptotic pathways, NFkB activation of inflammation, and activate stress-activated protein kinases (SAPKs). TNF-α receptors are present in both neurons and glia. TNF-α has been shown to play important roles in both inflammatory and neuropathic hyperalgesia. Intraplantar injection of complete Freund's adjuvant in adult rats resulted in significant elevation in the levels of TNF-α, IL-1β, and nerve growth factor (NGF) in the inflamed paw. A single injection of anti-TNF-α antiserum before the CFA significantly delayed the onset of the resultant inflammatory hyperalgesia and reduced IL-1β but not NGF levels. Intraplantar injection of TNF-α also produces mechanical and thermal hyperalgesia. It has been found that TNF-α injected into nerves induces Wallerian degeneration and generates the transient display of behaviors and endoneurial pathologies found in experimentally painful nerve injury. TNF binding protein (TNF-BP), an inhibitor of TNF, is a soluble form of a transmembrane TNF-receptor. When TNF-BP is administered systemically, the hyperalgesia normally observed after lipopolysaccharide (LPS) administration is completely eliminated. Intrathecal administration of a combination of TNF-BP and IL-1 antagonist attenuated mechanical allodynia in rats with L5 spinal nerve transection.

Rantes, also known as CCL5, is a chemoattractant for blood monocytes, memory T-helper cells and eosinophils. It causes the release of histamine from basophils and activates eosinophils. It may activate several chemokine receptors including CCR1, CCR3, CCR4 and CCR5. It is one of the major HIV-suppressive factors produced by CD8+ T-cells. Recombinant RANTES protein induces a dose-dependent inhibition of different strains of HIV-1, HIV-2, and simian immunodeficiency virus (SIV). The processed form RANTES (3-68) acts as a natural chemotaxis inhibitor and is a more potent inhibitor of HIV-1-infection. The second processed form RANTES (4-68) exhibits reduced chemotactic and HIV-suppressive activity compared with RANTES (1-68) and RANTES (3-68) and is generated by an unidentified enzyme associated with monocytes and neutrophils. Rantes may also be an agonist of the G protein-coupled receptor GPR75, stimulating inositol trisphosphate production and calcium mobilization through its activation. Together with GPR75, Rantes may play a role in neuron survival through activation of a downstream signaling pathway involving the PI3, Akt and MAP kinases. By activating GPR75 may also play a role in insulin secretion by islet cells.

Chemokines are a family of small cytokines, or signaling proteins secreted by cells. Their name is derived from their ability to induce directed chemotaxis in nearby responsive cells; they are chemotactic cytokines. In addition to being known for mediating chemotaxis, chemokines are all approximately 8-10 kilodaltons in mass and have four cysteine residues in conserved locations that are key to forming their 3-dimensional shape.

These proteins have historically been known under several other names including the SIS family of cytokines, SIG family of cytokines, SCY family of cytokines, Platelet factor-4 superfamily or intercrines. Some chemokines are considered pro-inflammatory and can be induced during an immune response to recruit cells of the immune system to a site of infection, while others are considered homeostatic and are involved in controlling the migration of cells during normal processes of tissue maintenance or development. Chemokines are found in all vertebrates, some viruses and some bacteria, but none have been described for other invertebrates.

Chemokines represent a family of low molecular weight secreted proteins that primarily function in the activation and migration of leukocytes although some of them also possess a variety of other functions. Chemokines have conserved cysteine residues that allow them to be assigned to four groups: C-C chemokines (monocyte chemoattractant protein or MCP-1, monocyte inflammatory protein or MIP-1α, and MIP-1β), C-X-C chemokines (IL-8 also called growth related oncogene or GRO/KC), C chemokines (lymphotactin), and CXXXC chemokines (fractalkine).

A growth factor is a naturally occurring substance capable of stimulating cellular growth, proliferation, healing, and cellular differentiation. Usually it is a protein or a steroid hormone. Growth factors are important for regulating a variety of cellular processes.

Growth factors typically act as signaling molecules between cells. Examples are cytokines and hormones that bind to specific receptors on the surface of their target cells. They often promote cell differentiation and maturation, which varies between growth factors. For example, epidermal growth factor (EGF) enhances osteogenic differentiation, while fibroblast growth factors (FGFs) and vascular endothelial growth factors (VEGFs) stimulate blood vessel differentiation (angiogenesis).

Platelet-derived growth factor (PDGF) is one among numerous growth factors that regulate cell growth and division. In particular, PDGF plays a significant role in blood vessel formation, the growth of blood vessels from already-existing blood vessel tissue, mitogenesis, i.e. proliferation, of mesenchymal cells such as fibroblasts, osteoblasts, tenocytes, vascular smooth muscle cells and mesenchymal stem cells as well as chemotaxis, the directed migration, of mesenchymal cells. Platelet-derived growth factor is a dimeric glycoprotein that can be composed of two A subunits (PDGF-AA), two B subunits (PDGF-BB), or one of each (PDGF-AB).

PDGF is a potent mitogen for cells of mesenchymal origin, including fibroblasts, smooth muscle cells and glial cells. In both mouse and human, the PDGF signalling network consists of five ligands, PDGF-AA through -DD (including -AB), and two receptors, PDGFRalpha and PDGFRbeta. All PDGFs function as secreted, disulphide-linked homodimers, but only PDGFA and B can form functional heterodimers.

Though PDGF is synthesized, stored (in the alpha granules of platelets), and released by platelets upon activation, it is also produced by other cells including smooth muscle cells, activated macrophages, and endothelial cells.

### Stem Cells

### Mesenchymal Stem Cell (MSC)

Mesenchymal stem cells (MSCs) also known as mesenchymal stromal cells or medicinal signaling cells are multipotent stromal cells that can differentiate into a variety of cell types, including osteoblasts (bone cells), chondrocytes (cartilage cells), myocytes (muscle cells) and adipocytes (fat cells which give rise to marrow adipose tissue). They can be isolated from various sources, such as bone marrow, adipose, synovial tissue, lung, molar tissue, amniotic fluid, umbilical cord blood, peripheral blood, and olfactory bulbs, or even in virtually all postnatal organs and tissues. Among these, the most frequently used MSCs in studies for stem cell-mediated repair (e.g., cardiac repair) are BM-derived MSC (BM-MSC) and adipose-derived MSC (ADSC).

MSCs are fairly heterogeneous cell population but lacks a specific marker to define MSCs. According to minimum criteria that were proposed by The International Society for Cell Therapy in 2006, MSCs are characterized as (1) adherence to plastic in standard culture conditions; (2) expressing surface molecules CD73, CD90, and CD105, but in the absence of f CD34, CD45, HLA-DR, CD14 or CD11b, CD79a, or CD19; (3) a capacity for differentiation to osteoblasts, adipocytes, and chondroblasts in vitro. Besides, MSCs possess species-specific characteristics, and the characteristics of MSCs may also vary according to the source of tissue. For example, ADSCs were superior to BMSC with respect to maintenance of proliferating ability.

### Dental Pulp Stem Cells

Dental pulp stem cells (DPSCs) are stem cells present in the dental pulp, which is the soft living tissue within teeth. They are pluripotent, as they can form embryoid body-like structures (EBs) in vitro and teratoma-like structures that contained tissues derived from all three embryonic germ layers when injected in nude mice. DPSCs can differentiate in vitro into tissues that have similar characteristics to mesoderm, endoderm and ectoderm layers. DPSCs were found to be able to differentiate into adipocytes and neural-like cells. These cells can be obtained from postnatal teeth, wisdom teeth, and deciduous teeth, providing researchers with a non-invasive method of extracting stem cells. As a result, DPSCs have been thought of as an extremely promising source of cells used in endogenous tissue engineering.

Studies have shown that the proliferation rate of DPSCs is 30% higher than in other stem cells, such as bone marrow stromal stem cells (BMSSCs). These characteristics of DPSCs are mainly due to the fact that they exhibit elevated amounts of cell cycling molecules, one being cyclin-dependent kinase 6 (CDK6), present in the dental pulp tissue. Additionally, DPSCs have displayed lower immunogenicity than MSCs.

Stem Cells from Human Exfoliated Deciduous Teeth (Shed)

Stem cells from human exfoliated deciduous teeth (SHED) are similar to DPSCs in the sense that they are both derived from the dental pulp, but SHED are derived from baby teeth, whereas DPSCs are derived from adult teeth. SHED are a population of multipotent stem cells that are easily collected, as deciduous teeth either shed naturally or are physically removed in order to facilitate the proper growth of permanent teeth. These cells can differentiate into osteocytes, adipocytes, odontoblast, and chondrocytes in vitro. Recent work has shown the enhanced proliferative capabilities of SHED when compared with that of dental pulp stem cells.

Studies have shown that under the influence of oxidative stress, SHED (OST-SHED) displayed increased levels of neuronal protection. The properties of these cells exhibited in this study suggest that OST-SHED could potentially prevent of oxidative stress-induced brain damage and could aid in the development of therapeutic tools for neurodegenerative disorders. After SHED injection into Goto-Kakizaki rats, type II diabetes mellitus (T2DM) was ameliorated, suggesting the potential for SHED in T2DM therapies.

Recent studies have also shown that the administration of SHED in mice ameliorated the T cell immune imbalance in allergic rhinitis (AR), suggesting the cells' potential in future AR treatments. After introducing SHED, mice experienced reduced nasal symptoms and decreased inflammatory infiltration. SHEDs were found to inhibit the proliferation of T lymphocytes, increase levels of an anti-inflammatory cytokine, IL-10, and decrease the levels of a pro-inflammatory cytokine, IL-4.

Additionally, SHED can potentially treat liver cirrhosis. In a study conducted by Yokoyama et al. (2019), SHED were differentiated into hepatic stellate cells. They found that when hepatic cells derived from SHED were transplanted into the liver of rats, liver fibrosis was terminated, allowing for the healing of the liver structure.

### Embryonic Stem Cell

Embryonic stem cells (ES cells or ESCs) are pluripotent stem cells derived from the inner cell mass of a blastocyst, an early-stage pre-implantation embryo. Human embryos reach the blastocyst stage 4-5 days post fertilization, at which time they consist of 50-150 cells. Isolating the embryoblast, or inner cell mass (ICM) results in destruction of the blastocyst. ESCs are distinguished by their ability to differentiate into any embryonic cell type and by their ability to self-renew. ESCs have a normal karyotype, maintain high telomerase activity, and exhibit remarkable long-term proliferative potential.

Embryonic stem cells of the inner cell mass are pluripotent, meaning they are able to differentiate to generate primitive ectoderm, which ultimately differentiates during gastrulation into all derivatives of the three primary germ layers: ectoderm, endoderm, and mesoderm. These germ layers generate each of the more than 220 cell types in the adult human body. When provided with the appropriate signals, ESCs initially form precursor cells that in subsequently differentiate into the desired cell types. Pluripotency distinguishes embryonic stem cells from adult stem cells, which are multipotent and can only produce a limited number of cell types.

Due to their plasticity and potentially unlimited capacity for self-renewal, embryonic stem cell therapies have been proposed for regenerative medicine and tissue replacement after injury or disease. Pluripotent stem cells have shown promise in treating a number of varying conditions, including but not limited to: spinal cord injuries, age related macular degeneration, diabetes, neurodegenerative disorders (such as Parkinson's disease), AIDS, etc. In addition to their potential in regenerative medicine, embryonic stem cells provide a possible alternative source of tissue/organs which serves as a possible solution to the donor shortage dilemma.

Induced Pluripotent Stem Cell (iPSC)

Induced pluripotent stem cells (also known as iPS cells or iPSCs) are a type of pluripotent stem cell that can be generated directly from a somatic cell. The iPSC technology was pioneered by Shinya Yamanaka's lab in Kyoto, Japan, who showed in 2006 that the introduction of four specific genes (named Myc, Oct3/4, Sox2 and Klf4), collectively known as Yamanaka factors, encoding transcription factors could convert somatic cells into pluripotent stem cells. He was awarded the 2012 Nobel Prize along with Sir John Gurdon “for the discovery that mature cells can be reprogrammed to become pluripotent.”

Pluripotent stem cells hold promise in the field of regenerative medicine. Because they can propagate indefinitely, as well as give rise to every other cell type in the body (such as neurons, heart, pancreatic, and liver cells), they represent a single source of cells that could be used to replace those lost to damage or disease.

Since iPSCs can be derived directly from adult tissues, they not only bypass the need for embryos, but can be made in a patient-matched manner, which means that each individual could have their own pluripotent stem cell line. These unlimited supplies of autologous cells could be used to generate transplants without the risk of immune rejection. The iPSCs are readily being used in personalized drug discovery efforts and understanding the patient-specific basis of disease.

### Hematopoietic Stem Cell

Hematopoietic stem cells (HSCs) are the stem cells that give rise to other blood cells. This process is called haematopoiesis. This process occurs in the red bone marrow, in the core of most bones. In embryonic development, the red bone marrow is derived from the layer of the embryo called the mesoderm.

Haematopoiesis is the process by which all mature blood cells are produced. It must balance enormous production needs (the average person produces more than 500 billion blood cells every day) with the need to regulate the number of each blood cell type in the circulation. In vertebrates, the vast majority of hematopoiesis occurs in the bone marrow and is derived from a limited number of hematopoietic stem cells that are multipotent and capable of extensive self-renewal.

Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells. The definition of hematopoietic stem cell has developed since HSCs were first discovered in 1961. The hematopoietic tissue contains cells with long-term and short-term regeneration capacities and committed multipotent, oligopotent, and unipotent progenitors. Hematopoietic stem cells constitute 1:10,000 of cells in myeloid tissue.

HSC transplants are used in the treatment of cancers and other immune system disorders. For example, HSC transplants are often performed for patients with certain cancers of the blood or bone marrow, such as multiple myeloma or leukemia. In these cases, the recipient's immune system is usually destroyed with radiation or chemotherapy to remove cancerous cells before the transplantation with non-cancerous HSCs. Cancer indications for HSC transplant include acute myeloid leukemia, chronic myeloid leukemia, acute lymphoblastic leukemia, hodgkin lymphoma, non-Hodgkin lymphoma, neuroblastoma, Ewing sarcoma, multiple myeloma, myelodysplastic syndromes, and gliomas.

### Exemplary Diseases

The compositions and methods described herein can be used in the treatment of a subject in need of stem cell transplantation. In some embodiments, the subject is afflicted with a neurodegenerative disease, paralysis, cancer (e.g., leukemia, lymphoma, multiple myeloma), liver cirrhosis, or ischemic heart disease. In some embodiments, the disease is related to neurodegeneration and/or neuromuscular disorders. Representative such conditions are described below.

### Ischemic Heart Disease

Ischemic heart disease is the leading cause of death worldwide. Severe ischemic heart disease, especially myocardial infarction (MI) and heart failure, causes a significant loss of functional cardiomyocytes. However, heart is an organ with very limited self-renewal capacity because adult cardiomyocytes can hardly regenerate. Over the past decades, there has been tremendous enthusiasm in an attempt to repair cardiac tissue with stem cell transplantation. Mesenchymal stem cell (MSC), with advantages in immunologic privilege, easy to be acquired, and multilineage potential, has been widely studied both in animal model and in clinical trials. Low survival rate after transplantation is one of the crucial reasons accounting for the hampered cardiac repair effect of MSC. The harsh microenvironment with ischemia, inflammation, oxidative stress, and mechanical stress contributes to the great cell loss.

The first study exploring the cardiac regenerative effect of MSC was carried out in 1999 on a rat MI model induced by cryoinjury. The autologous MSC was induced into cardiogenic cells by 5-azacytidine in vitro and transplanted into the scar of the injured hearts. The transplantation improved cardiac function, prevented remodeling, and promoted angiogenesis. In the following decades, MSCs were transplanted for treating chronic or acute ischemic heart injury in rodent models and large animals. The underlying mechanisms for the therapeutic effect include directly transdifferentiation into functional cardiomyocyte/endothelial cell, secretion of a broad spectrum of cytokine in a paracrine manner, and stimulating local cardiac stem cell proliferation. It was reported that MSC can differentiate into cardiomyocyte phenotype induced by 5-azacytidine, coculture, and in vivo models.

Neurodegeneration Neurodegeneration is the progressive loss of structure or function of neurons, including death of neurons. Many neurodegenerative diseases—including amyotrophic lateral sclerosis, Parkinson's disease, Alzheimer's disease, and Huntington's disease—occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously.

### Alzheimer's Disease

Alzheimer's disease is a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioural issues. As a person's condition declines, they often withdraw from family and society. Gradually, bodily functions are lost, ultimately leading to death. Although the speed of progression can vary, the typical life expectancy following diagnosis is three to nine years.

The cause of Alzheimer's disease is poorly understood. About 70% of the risk is believed to be inherited from a person's parents with many genes usually involved. Other risk factors include a history of head injuries, depression, and hypertension. The disease process is associated with plaques and neurofibrillary tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis. There are no medications or supplements that have been shown to decrease risk.

No treatments stop or reverse its progression, though some may temporarily improve symptoms. Affected people increasingly rely on others for assistance, often placing a burden on the caregiver. The pressures can include social, psychological, physical, and economic elements. Exercise programs may be beneficial with respect to activities of daily living and can potentially improve outcomes. Behavioural problems or psychosis due to dementia are often treated with antipsychotics, but this is not usually recommended, as there is little benefit with an increased risk of early death.

In 2015, there were approximately 29.8 million people worldwide with Alzheimer's disease. It most often begins in people over 65 years of age, although 4-5% of cases are early-onset Alzheimer's. In 2015, dementia resulted in about 1.9 million deaths. In developed countries, Alzheimer's disease is one of the most financially costly diseases.

### Parkinson's Disease

Parkinson's disease is a long-term degenerative disorder of the central nervous system that mainly affects the motor system. As the disease worsens, non-motor symptoms become more common. The symptoms usually emerge slowly. Early in the disease, the most obvious symptoms are shaking, rigidity, slowness of movement, and difficulty with walking. Thinking and behavioral problems may also occur. Dementia becomes common in the advanced stages of the disease. Depression and anxiety are also common, occurring in more than a third of people with Parkinson's disease. Other symptoms include sensory, sleep, and emotional problems.

The cause of Parkinson's disease is unknown, but is believed to involve both genetic and environmental factors. Those with a family member affected are more likely to get the disease themselves. There is also an increased risk in people exposed to certain pesticides and among those who have had prior head injuries, while there is a reduced risk in tobacco smokers and those who drink coffee or tea. The motor symptoms of the disease result from the death of cells in the substantia nigra, a region of the midbrain. This results in not enough dopamine in this region of the brain. The cause of this cell death is poorly understood, but it involves the build-up of proteins into Lewy bodies in the neurons.

There is no cure for Parkinson's disease. Treatment aims to improve the symptoms. Initial treatment is typically with the antiparkinson medication levodopa (L-DOPA), followed by dopamine agonists when levodopa becomes less effective. As the disease progresses and neurons continue to be lost, these medications become less effective while at the same time they produce a complication marked by involuntary writhing movements. Diet and some forms of rehabilitation have shown some effectiveness at improving symptoms. Surgery to place microelectrodes for deep brain stimulation has been used to reduce motor symptoms in severe cases where drugs are ineffective.

In 2015, Parkinson's disease affected 6.2 million people and resulted in about 117,400 deaths globally. Parkinson's disease typically occurs in people over the age of 60, of whom about one percent are affected. Males are more often affected than females at a ratio of around 3:2. When it is seen in people before the age of 50, it is called early-onset PD. The average life expectancy following diagnosis is between 7 and 15 years.

### Huntington's Disease

Huntington's disease, also known as Huntington's chorea, is an inherited disorder that results in the death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline into dementia. The specific symptoms vary somewhat between people. Symptoms usually begin between 30 and 50 years of age, but can start at any age. The disease may develop earlier in life in each successive generation. About eight percent of cases start before the age of 20 years and typically present with symptoms more similar to Parkinson's disease.

Huntington's disease is typically inherited, although up to 10% of cases are due to a new mutation. The disease is caused by an autosomal dominant mutation in either of an individual's two copies of a gene called Huntingtin. This means a child of an affected person typically has a 50% chance of inheriting the disease. The Huntingtin gene provides the genetic information for a protein that is also called “huntingtin.” Expansion of CAG (cytosine-adenine-guanine) triplet repeats in the gene coding for the Huntingtin protein results in an abnormal protein, which gradually damages cells in the brain, through mechanisms that are not fully understood.

There is no cure for Huntington's disease. Treatments can relieve some symptoms and in some improve quality of life. The best evidence for treatment of the movement problems is with tetrabenazine. Huntington's disease affects about 4 to 15 in 100,000 people of European descent. The disease affects men and women equally. Complications such as pneumonia, heart disease, and physical injury from falls reduce life expectancy. Suicide is the cause of death in about 9% of cases. Death typically occurs fifteen to twenty years from when the disease was first detected.

### Batten Disease

Batten disease is a fatal disease of the nervous system that typically begins in childhood. Onset of symptoms is usually between 5 and 10 years of age. Often, it is autosomal recessive. It is the most common form of a group of disorders called the neuronal ceroid lipofuscinoses (NCLs). At least 20 genes have been identified in association with Batten disease, but juvenile NCL, the most prevalent form of Batten disease, has been linked to mutations in the CLN3 gene.

### Muscle Atrophy: Loss of Muscle Function and Muscle Mass Due to Denervation

When the information transfer between the nerve cells and the skeletal muscles is impaired or even disrupted, as it happens with degeneration of motor neurons that gradually leads to denervation, the muscles can no longer function properly and begin to become atrophic.

Denervation is an injury to the peripheral neurons with a partial or completion interruption of the nerve fibers between an organ and the central nervous system, resulting in an interruption of nerve conduction and motoneuron firing which, in turn, prevents the contractability of skeletal muscles. This loss of nerve function can be localized or generalized due to the loss of an entire motor neuron unit. The resulting inability of skeletal muscles to contract leads to muscle atrophy; within only a few week weeks, a major part of the muscle mass can be lost, as evidenced by a decrease in muscle weight as well as muscle function.

Muscle Atrophy Resulting from Traumatic Nerve Injury and Neurodegenerative Motoneuron Diseases

Muscle atrophy severely affects the quality of life, as the concerned individuals are impaired or even incapable of performing tasks that involve lifting, walking or running. In motoneuron diseases, the information transmission from motor neurons in the spinal cord to skeletal muscle fibers via somatic motor nerve fibers is impaired or fully interrupted. Motor neurons and muscle fibers interface at the neuromuscular junction. Upon stimulation in vertebrates, the motor neuron releases neurotransmitters that bind to postsynaptic receptors and trigger an excitatory, i.e. contractile, response in the muscle fiber. Since, thus, the contraction of a skeletal muscle can only be prompted through the firing of motor neurons with the transmission of a nerve impulse, an interruption of that transmission means that the skeletal muscle becomes inactive and atrophic over time. The interruption of nerve function can occur in the brain, spinal cord, or a peripheral nerve.

### Motoneuron Diseases

Motoneuron diseases are neurological disorders that selectively and irreversibly destroy motoneurons, the cells that control voluntary muscle activity such as speaking, walking, breathing, swallowing and general movement of the body. Motoneuron diseases are primarily inherited and occur in children as well as adults; they are classified in accordance to whether they affect upper motor neurons, lower motor neurons or both. Motoneuron diseases are generally progressive in nature, and cause gradually increasing disability and death.

### Amyotrophic Lateral Sclerosis.

Amyotrophic lateral sclerosis (Lou Gehrig's Disease; ALS), also known as motor neurone disease, is considered the most common form of a motoneuron disease with an onset in adult age of, in average, about 50-60 years and an incidence of 1:50,000 per year.

ALS results in the death of neurons controlling voluntary muscles. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and most people experience pain. Most eventually lose the ability to walk, use their hands, speak, swallow, and breathe. ALS is a progressive disease with a fatal outcome due to gradual paralysis of all voluntary muscles throughout the body, whereby the breathing and swallowing muscles become affected early on already.

The cause is not known in 90% to 95% of cases, but is believed to involve both genetic and environmental factors. The remaining 5-10% of cases are inherited from a person's parents. The most common familial forms of ALS in adults are caused by mutations of the superoxide dismutase gene, or SOD1, located on chromosome 21. The underlying mechanism involves damage to both upper and lower motor neurons.

No cure for ALS is known. The goal of current treatment is to improve symptoms. A medication called riluzole may extend life by about two to three months. Non-invasive ventilation may result in both improved quality and length of life. Mechanical ventilation can prolong survival but does not stop disease progression. A feeding tube may help. The disease can affect people of any age, but usually starts around the age of 60 and in inherited cases around the age of 50. The average survival from onset to death is two to four years, though this can vary. About 10% survive longer than 10 years. Most die from respiratory failure.

### Liver Cirrhosis

Liver cirrhosis is a condition where scar tissue replaces the healthy tissue of the liver and regenerative nodules with surrounding fibrous bands develop as a result of the injury. Cirrhosis is the common end of progressive liver disease of various causes, resulting in chronic liver failure entailing complications such as hepatic encephalopathy, spontaneous bacterial peritonitis, ascites, and esophageal varices. Unfortunately, the majority of cases are usually in an irreversible state when diagnosed. Despite current advancements in its management, cirrhosis was the 14th leading cause of death worldwide in 2012. Orthotopic liver transplantation is known to be the only definite solution to end-stage cirrhosis, but several problems preclude the prevalent application of the procedure, including immunological rejection and the scarcity of donor sources.

The transplantation of stem cells has shown therapeutic potential for liver function improvement according to recent experimental studies and human studies. Although they remain unclear, the major potential mechanisms have been proposed as a twofold; one is the improvement of the microenvironments through paracrine effects, and the other is the replacement of functional hepatocytes. Multiple types of stem cells are known to differentiate into hepatocyte-like cells (HLCs): hematopoietic stem cells (HSCs), mesenchymal stem cells (MSCs), embryonic stem cells (ESCs), induced pluripotent stem cells (iPSCs), and endothelial progenitor cells (EPCs). Early infusion trials showed improved liver function, irrespective of the origins of the stem cells.

### Cancer

As described herein, the methods and compositions provided herein can be used for preventing or treating cancer.

Cancer, tumor, or hyperproliferative disease refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, (e.g., multiple myeloma, Diffuse large B-cell lymphoma (DLBCL), Follicular lymphoma, Chronic lymphocytic leukemia (CLL), small lymphocytic lymphoma (SLL), Mantle cell lymphoma (MCL), Marginal zone lymphomas, Burkitt lymphoma, Waldenstrom's macroglobulinemia, Hairy cell leukemia, Primary central nervous system (CNS) lymphoma, Primary intraocular lymphoma, the heavy chain diseases, such as, for example, alpha chain disease, gamma chain disease, and mu chain disease, benign monoclonal gammopathy, and immunocytic amyloidosis), T cell cancer (e.g., T-lymphoblastic lymphoma/leukemia, non-Hodgkin lymphomas, Peripheral T-cell lymphomas, Cutaneous T-cell lymphomas (e.g., mycosis fungoides, Sezary syndrome), Adult T-cell leukemia/lymphoma, Angioimmunoblastic T-cell lymphoma, Extranodal natural killer/T-cell lymphoma, Enteropathy-associated intestinal T-cell lymphoma (EATL), Anaplastic large cell lymphoma (ALCL), Hodgkin lymphoma), melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

Exemplary Additional Agents or Precondition that Enhance the Survival of Stem Cells

### Biomaterials

Due to the disrupted extracellular cell matrix (ECM) and compressive mechanical stress, the target tissue (e.g., infarcted myocardium) is not an environment conducive to cell survival. Scaffolds temporarily provide the biomechanical support for cells until they are able to produce their own extracellular matrix. For example, scaffolds seeded with MSC showed better performance in cardiac repair than injection of MSC alone. There are mainly two types of scaffold: hydrogel and cell sheet (Li et al. (2016) Stem Cells Int. 9682757:1-14).

### Thermosensitive Hydrogel

Hydrogel as a biocompatible material was used to prevent the first wave loss of transplanted MSC due to the myocardium contraction. Hydrogels are in situ formation, biodegradable, and cell adhesive. Once delivered together with the stem cells, it can self-cross-link to form semigrid scaffold which could ameliorate the cell loss.

There are various types of hydrogels applied in stem cell therapy: (1) natural hydrogels, such as fibrin glue, collagen, alginate, and cardiogel. Cardiogel is a cardiac fibroblast-derived ECM, which was designed to mimic the natural environment suitable for transplanted MSC; (2) synthetic hydrogel, including silanized hydroxypropyl methylcellulose (Si-HPMC) and poly(lactide-co-epsilon-caprolactone); (3) combination of different materials in a certain ratio, such as poly(N-isopropylacrylamide) (PNIPAAm) plus single-wall carbon nanotubes (SWCNTs), alginate/chitosan, poly(glycerol sebacate) combined with collagen, and hydrophobic poly(F-caprolactone)-2-hydroxylethyl methacrylate (PCL-HEMA) plus PNIPAAm. Hydrogel can also serve as a medium to support the diffusion of molecules. Since interleukin-10 (IL-10) is an anti-inflammation cytokine, a combination of MSC, Matrigel, and IL-10 plasmids was designed to improve cell survival.

Hydrogel is effective in improving cell survival in stem cell therapy. For example, in a rat myocardial infarction (MI) model, intramyocardial injection of MSCs with Si-HPMC (one of the synthetic hydrogels) showed better performance in cell retention and cardiac function preservation than MSCs injection alone. In a swine MI model, retention of MSC suspended in 2% alginate (a natural hydrogel) before transplantation was approximately 4-fold compared to that in control MSCs at two weeks after delivery. Similarly, coinjection with fibrin glue increased ADSC survival by about 30% on a rat MI model.

The first clinical trial using injectable bioabsorbable scaffold (IK-5001), a solution of 1% sodium alginate plus 0.3% calcium gluconate, combined with MSCs by intracoronary delivery has been carried out (world wide web at clinicaltrials.gov.: NCT01226563). This first-in-man pilot study also showed that intracoronary deployment of an IK-5001 scaffold is feasible, effective, and well tolerated in patients with STEMI.

### Patch Cell Sheet

To avoid the shortcomings of needle injection, biocompatible patches seeded with MSC emerged as an alternative strategy to circumvent the lack of cell engraftment. Solid form of biomaterials (such as collagen) seeded with cells was sutured onto the surface of infarcted area. The patch can be absorbed gradually while the stem cells engrafted into the myocardium.

For example, ADSC-cellularized sheets were implanted onto the epicardium of on chronic rat MI model. No cell was detected in ADSC alone group, but cell sheet exhibited 25.3±7.0% and 6.4±4% engraftment rate at 1 week and 1 month after MI. The same group performed a head-to-head comparison of cell engraftment between the conventional injection, deposition of the bilayer myoblast cell sheet, and deposition of the myoblast cells seeded in collagen sponge in rat MI model. Both cell constructs are superior to conventional needle injection. The myoblast-seeded collagen sponge group produced the best outcome with regard to engraftment cells number and reduced fibrosis.

### Hypoxic, Hyperoxic, and Pharmacological Preconditioning of MSCS

Although severe hypoxia can lead to cell death, repeated episodes of short period exposure to hypoxia (hypoxia-preconditioning) have shown conferring cytoprotective benefits (Li et al. (2008) J of Endocrinological investigation. 31(2):103-110). Stem cells were cultured under hypoxia (0.5% oxygen) or normoxic conditions for 24 hours: hypoxia-preconditioning reduced about 25% of cell death at day 1 and 40% of cell death at day 3 after delivery compared with normoxic control. This effect is associated with the increased expression of prosurvival and proangiogenic factors including hypoxia-inducible factor 1 (HIF-1α), angiopoietin-1, vascular endothelial growth factor (VEGF), erythropoietin, Bcl-2, and Bcl-xL. Moreover, hypoxia-preconditioning induced autophagy protected the stem cells from apoptosis, which may be also accounted for the improvement of stem cell survival.

Preconditioning with hyperoxia (100% oxygen) or/and Z-VAD-FMK pan-caspase inhibitor also promoted stem cell viability and proliferation, but by a different mechanism of decreasing caspases 1, 3, 6, 7, and 9 expression and increasing survival genes such as Akt.

Sevoflurane, an inhaled anesthetic widely used in clinical anesthesia, has similar effect of hypoxia-preconditioning. Sevoflurane pretreatment minimized stem cell apoptosis and the loss of its mitochondrial membrane potential induced by hypoxia, which may be mediated by HIF and Akt pathways.

Studies also revealed that stem cells for transplantation could be preconditioned by coculturing with cells. For example, MSC preconditioned with cardiomyocytes in culture exerted enhanced therapeutic effect compared with MSC alone. The hetero-cell-to-cell connection altered the MSC paracrine of cardioprotective soluble factors such as VEGF, HGF, SDF-1α, and MCP-3.

Preconditioning of stem cells with TGF-α enhanced the VEGF secretion of transplanted stem cells in vivo. Platelet-derived growth factor-BB (PDGF) treatment of stem cells resulted in rapid activation of both Akt and ERK and upregulated VEGF. Thus, stem cells with PDGF preconditioning exhibited a greater capacity of functional recovery.

Preconditioning of stem cells with an inhibitor of Janus kinase (JAK) and/or an inhibitor of p38 also improve the stem cell survival (U.S. Patent Publication No. 2008/0242594). Exemplary JAK/STAT inhibitors are known in the art (U.S. Patent Publication No. 2004/0209799).

Importantly, preconditioning can be operated in vitro prior to transplantation.

In some embodiments, the stem cells are preconditioned as described herein for at least, about, or no more than 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, or 24 hours. In some embodiments, the stem cells are preconditioned as described herein for at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

### Other Exemplary Therapies for Neurodegenerative Disorders

There is no treatment that can cure neurodegenerative diseases, but there are symptomatic treatments. These include dopaminergic treatments for Parkinson's disease and movement disorders, cholinesterase inhibitors for cognitive disorders, antipsychotic drugs for behavioral and psychological symptoms of dementia, analgesic drugs for pain, and even the use of deep brain stimulation to stop tremor and refractory movement disorders. Researchers have also aimed to produce medicines to slow the development of diseases, such as Riluzole for ALS, cerebellar ataxia and Huntington's disease, NSAIDs (nonsteroidal anti-inflammatory drugs) for Alzheimer's disease, and caffein A2A receptor antagonists and CERE-120 (adeno-associated virus serotype 2-neurturin) for the neuroprotection of Parkinson's disease.

In some embodiments, a condition such as neurodegenerative disorder is responsive to blockade of cytokines, chemokines, and/or growth factors alone. In certain such embodiments, the condition is significantly or synergistically more responsive when treated in combination with another therapy.

### Alzheimer's Disease (AD)

Currently, there is no known cure for AD, and the drugs used within the scope of this disease are mainly to treat the cognitive manifestations or other symptoms and function better when administered at an early stage. One of the drugs that is marketed for the treatment of AD, galantamine was repurposed. In fact, this alkaloid, present in Galanthus sp., aroused interest when it was found that it could inhibit muscle acetylcholinesterase, being a good candidate for treating myopathies and peripheral neuropathies, and for the reversal of neuromuscular blockade after anaesthesia, due to the capability of galantamine to enhance nerve impulse transmission. As it has a tertiary ammonium base, galantamine can easily penetrate the blood-brain barrier and inhibit brain acetylcholinesterase.

Antimicrobials have also been studied for their potential suitability to treat AD and their symptoms. Both azithromycin and erythromycin, macrolide antibiotics, have shown inhibition of the amyloid precursor protein, resulting in the decrease of cerebral levels of amyloid-β. Tetracyclines have also been proven to reduce the formation of amyloid-β, as well as its resistance to trypsin digestion and an increase in the disassembly of preformed fibrils. They also decreased oxidative stress, suggesting a varied mechanism of action. Doxycycline has shown potential in this respect, both alone and in combination with rifampicin. Rifampicin, most frequently prescribed for Mycobacterium infections, has shown effects in the reduction of amyloid-β fibrils, in a dose-dependent manner, probably due to the decreased production and increased clearance of amyloid-β. Dapsone is an antibiotic used to treat leprosy, and also received attention when a decreased incidence of dementia was noticed in leprosy patients that had been treated with dapsone. Conflicting data about whether or not dapsone was capable of decreasing senile plaques led to the hypothesis that this event could be a protective factor against amyloid deposition. This hypothesis was further corroborated by studies that showed similar instances of AD in leprosy and tuberculosis patients, in spite of the differences in the percentage of patients that have undergone drug treatments in the two groups. The antiviral drugs acyclovir, penciclovir and foscarnet have been successful in reducing phosphorylated tau protein and amyloid-β in AD cell models, which can mean they are suitable for the treatment of AD. Clioquinol is an antifungal and antiparasitic drug that has been shown to cause a reduction in the amyloid-β plaques in the brain, with good tolerability in transgenic mice.

The antiepileptic drug valproic acid has been suggested as a neuroprotective agent for AD, as it has shown reduced formation of amyloid-β plaques and improvement in memory deficits in transgenic mice. The proposed mechanism of action was shown to be complex, but it might be through the enhancement of microglial phagocytosis of amyloid-β.

Valsartan is an angiotensin receptor blocker, and is used as an antihypertensive. The rationale behind the use of this class of drugs for AD comes from the fact that chronic adverse stress, one of the major environmental causes for the onset and progression of AD, is capable of causing elevations in brain angiotensin II, which act at AT1 and AT2 receptor subtypes. Furthermore, angiotensin II increases have been suggested to be linked with amyloidogenesis, and the use of angiotensin receptor blockers, blocking AT1, appears to be useful in delaying decline in cognitive processing. Apart from this mechanism of action, valsartan also inhibits inflammation, vasoconstriction and mitochondrial dysfunction, and promotes the release of acetylcholine. Reduced amyloid-β has been reported with in vitro and in vivo treatment of valsartan, and this evidence suggests a reduction of dementia. Additionally, this drug has good brain penetration, but further studies are required before this drug can be included in the therapy of AD. Calcium channel blockers are drugs used to treat hypertension and angina. The dihydropyridine calcium channel blockers, such as nilvadipine, can reduce the production, oligomerization and accumulation of amyloid-β in vitro, improve cell survival and reduce neurotoxicity, while having good blood-brain barrier penetration and increasing brain blood flow through its vasodilatory properties.

Trimetazidine is an anti-ischemic drug of the piperazine class. Its mechanism of action is diverse, ranging from increasing nitric oxide production, inhibiting cell apoptosis and being an antioxidant, which increases endothelial function. Apart from being able to pass through the blood-brain barrier, it can reduce the produce of free radicals, due to its antioxidant properties. It can also improve axonal regeneration and effective myelination in healthy and injured nerves.

Antidiabetics have also been repurposed for AD, since type 2 diabetes has been identified as a risk factor for AD. Studies have reported a desensitization of insulin signalling in the brains of AD patients. Insulin can also induce neuronal stem cell activation and cell growth and repair, and treatment with insulin has shown neuroprotection and a regulation on the levels of phosphorylated tau protein, as well as an improvement in memory and cognition. Given this, compounds that influence insulin release can also be useful for AD. Glucagon-like peptide 1 analogues, which promote insulin secretion, may also act in many pathways related to AD, such as the reduction of amyloid-β and the impairment of neuronal function and cell death, as well as tau phosphorylation. Liraglutide meets these criteria, has established brain penetration and shows physiological effects in the brain, improving learning, and reducing amyloid-β formation and brain inflammation.

Other therapies include Ghrelin, hexarelin and its derivative EP80317, retinoid receptor activators, retinoic acid, zileuton (a drug that acts through the blockage of 5-lipooxygenase), sildenafil, tadalafil, and trazodone.

### Parkinson's Disease (PD)

Nilotinib is a tyrosine kinase Abl inhibitor that is used for the treatment of chronic myeloid leukaemia. It was observed that Abl is activated in neurodegeneration through the increase in α-synuclein expression and, therefore, its accumulation. Since nilotinib inhibits Abl phosphorylation, it increases α-synuclein degradation.

Zonisamide is a sulphonamide antiepileptic drug, with a mixed mechanism of action, which makes it appropriate for use in different disorders. These mechanisms of action include the blockage of sodium and calcium channels, modulation of the GABAA receptor, inhibition of carbonic anhydrase and inhibition of glutamate release. Studies with rats have shown an increase in dopamine in the striatum when therapeutic doses were used. On the other hand, when higher doses were used, a decrease in intracellular dopamine was observed. Concerning PD, this drug has displayed good activity in both motor and non-motor symptoms, but the mechanism of action is still unclear. Zonisamide is also a monoamine oxidase-B inhibitor. This enzyme, mostly present in astrocytes, is responsible for the degradation of dopamine in neural and glial cells, which ultimately leads to the generation of free radicals, which can play a determinant role in the pathogenesis of PD. Its inhibition makes dopamine levels in the synaptic cleft stable and increases the effect of dopamine.

Methylphenidate is a central nervous system stimulant that acts through the blockage of the presynaptic dopamine transporter and the noradrenaline transporter, thus inhibiting dopamine and noradrenaline reuptake, in the striatum and the prefrontal cortex. It has been used to treat attention-deficit hyperactivity disorder. Multiple studies with this drug have shown that it is effective in reducing gait disorders of PD, as well as non-motor symptoms.

β2-adrenoreceptor agonists, have been studied for their anti-PD activity. Recent findings have linked the β2-adrenoreceptor with the regulation of the α-synuclein gene SNCA. More specifically, β2-adrenoreceptor activation was shown to display neuroprotection. From the drugs tested, three anti-asthmatics were the most promising, with salbutamol being the one capable of penetrating the blood-brain barrier and currently approved for treatment. The study undertaken showed that all three drugs were able to reduce the SNCA-mRNA and α-synuclein abundance.

### Huntington's Disease (HD)

Tetrabenazine was first developed as part of research aiming to design simple compounds with reserpine-like antipsychotic activity, acting as a high-affinity, reversible inhibitor of monoamine uptake of presynaptic neurons, and as a weak blocker of the D2 dopamine postsynaptic neurons. Antipsychotic studies with this compound were equivocal, and this drug was then repurposed for diseases that manifest themselves by abnormal, involuntary hyperkinetic movements, such as HD. Furthermore, tetrabenazine is safer to use in HD than dopamine receptor blocker, since it has never been documented to cause dyskinetic symptoms. Given this, other drugs with dopamine antagonistic activity have been tested for the treatment of HD. This is the case of tiapride, a D2 receptor antagonist, used as an antipsychotic. However, in Europe, selegiline is a frequent choice for the treatment of Huntington's chorea. Clozapine is a neuroleptic drug used in the treatment of schizophrenia. It displays a high affinity for the dopamine D1 and D4 receptors, with low antagonistic activity for the D2 dopaminergic receptors. Due to its low incidence of extrapyramidal side effects, it was suggested to be a good symptomatic drug for chorea, although clinical trials showed conflicting results. Olanzapine, another antipsychotic drug, is also widely prescribed for the treatment of the motor and behavioural symptoms of HD. This drug has high affinity for serotoninergic receptor, but antagonizes dopamine D2 receptors. It is also safe and well tolerated, and can be recommended when irritability, sleep dysfunction and weight loss are present, as well as chorea. The antipsychotic risperidone, used in the treatment of schizophrenia and bipolar disorder, acts as a D2 receptor antagonist and a serotonin agonist, and therefore can be used for the treatment of HD chorea, as well. It showed beneficial effects on stabilizing motor decline and psychiatric symptoms.

Memantine is an adamantane derivative used for the treatment of AD. It is a non-competitive N-methyl-d-aspartate (NMDA) inhibitor. Excessive stimulation of NMDA receptor causes a great influx of calcium into the cell, which ultimately leads to cell death. Therefore, memantine can prevent this calcium influx in neuronal cells, and prevent cerebral cell death. Memantine was studied for its efficacy in the treatment of HD, and it was noticed that it was able to decrease the vulnerability of neurons to glutamate-mediated excitotoxicity.

### Multiple Sclerosis (MS)

Wide arrays of anticancer drugs have been repurposed for the treatment of MS and its symptoms. This is the case of the synthetic compounds mitoxantrone, an anthracenedione, established as a wide-spectrum antitumor agent used to treat breast and prostate cancer, acute leukaemia and lymphoma. Mitoxantrone has also been approved for the treatment of MS, particularly due to its immunosuppressant properties, associated with erratic responses of the central nervous system T- and B-cells to antigens, myelin damage mediated by macrophages, and axonal injuries. Mitoxantrone is capable of inhibiting the activation of T-cells, stopping the proliferation of T- and B-cells, lowering antibody production and deactivating macrophages. Mitoxantrone also displayed high tolerability. The alkylating agent cyclophosphamide is used to treat a variety of solid tumours, and is approved for the treatment of leukaemia, lymphomas, and breast carcinoma, among others. It is related to nitrogen mustards and binds to DNA, interfering with mitosis and cell replication, targeting mostly rapidly dividing cells. Its use in MS comes from cyclophosphamide being able to play an immunosuppressive and immunomodulatory role. Explicitly, it acts in T- and B-cells, suppressing cell-mediated and humoral immunity. Cyclophosphamide can also permeate the blood-brain barrier, having a good bioavailability in the central nervous system, being able to exert its activity on neurons, thus stabilizing and preventing the progression of the disease.

Amiloride is a diuretic drug used to treat hypertension and swelling caused by heart failure or liver diseases. It has been studied for its neuroprotective properties in MS. Amiloride can block the neuronal proton-gated acid-sensing ion channel 1 (ASIC1), which is overexpressed in axons and oligodendrocytes in MS lesions, thus exerting its neuroprotective and myeloprotective effects.

The drug ibudilast was approved in some countries for the treatment of bronchial asthma and cerebrovascular disorders. It acts through the inhibition of phosphodiesterases, but can also inhibit leukotriene and nitric oxide synthesis mechanisms, which are connected to MS. In the brain, ibudilast can inhibit the release of the tumour necrosis factor from the microglia and the astrocytes, decreasing neuronal degeneration. Furthermore, it can protect astrocytes from apoptosis and inhibit oligodendrocyte apoptosis and demyelination, hence its usefulness in MS. Studies have shown its safety and tolerability, while reducing the rate of brain atrophy at a high dose.

### Amyotrophic Lateral Sclerosis (ALS)

Only two drugs, riluzole and edaravone, are currently available to delay the progression of the disease, although they cannot revert the symptoms once they have manifested. Riluzole prolongs ALS survival; it increases survival rates at 12 months by 10% and prolongs survival by 6 months. Similarly Edaravone is effective in treating ALS.

Masitinib is a tyrosine kinase inhibitor used to treat cancer in dogs. Its use in ALS resides in the fact that abnormal glial cells that proliferate in ALS might be sensitive to tyrosine kinase inhibitors. It was proven that mastinib inhibited glial cell activation in the appropriate rat model and increased survival.

Retigabine is an approved drug for epilepsy, and acts by binding to the voltage-gated potassium channels and increasing the M-current, thus leading to membrane hyperpolarization. Retigabine is able to prolong motor neuron survival and decrease excitability, which is advantageous in the treatment of ALS, since it is believed that, in this disease, neurons are hyper-excitable, firing more than normal and ultimately leading to cell death. This drug is still under clinical trial for the treatment of ALS.

Tamoxifen is an antioestrogen drug, approved for the chemotherapy and chemoprevention of breast cancer. The repurposing of this drug for the treatment of ALS arose serendipitously, after the observation of a neurological improvement in patients and disease stabilization in ALS patients with breast cancer treated with tamoxifen. Its neuroprotective properties appear to be related to inhibition of protein kinase C, which is overexpressed in the spinal cord of ALS patients. Moreover, tamoxifen was found to be able to modulate a proteinopathy present in ALS, through its capacity to be an autophagy modulator.

### Other Exemplary Therapies for Cancer

The compositions and methods of the present invention can be used alone (e.g., for bone marrow transplant) or can be administered in combination therapy with, e.g., chemotherapeutic agents, hormones, antiangiogens, radiolabelled, compounds, or with surgery, cryotherapy, and/or radiotherapy. The treatment methods can be administered in conjunction with other forms of conventional therapy (e.g., standard-of-care treatments for cancer well-known to the skilled artisan), either consecutively with, pre- or post-conventional therapy. For example, compositions of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the composition is administered in combination with one or more inhibitors of immune checkpoints, such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with the pharmaceutical composition of the present disclosure to treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

The immunotherapy and/or cancer therapy may be administered before, after, or concurrently with the compositions described herein. The duration and/or dose of treatment with the composition may vary according to the particular composition, or the particular combinatory therapy. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

In some preferred embodiments, at least one cancer therapy is used to treat the subject before the transplantation of the composition comprising monocytes and stem cells.

### Pharmaceutical Compositions

The compositions of the present disclosure and/or additional therapeutic agent can be incorporated into pharmaceutical compositions suitable for administration to a subject.

For pharmaceutical compositions comprising the cells of the present disclosure, cells (e.g., monocytes and/or stem cells) can be administered at a dose of at least about 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, or more, or any range in between or any value in between, cells per kilogram of subject body weight.

The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiments, transplantation of at least about 100, 1000, 10,000, 0.1×10⁶, 0.5×10⁶, 1.0×10⁶, 2.0×10⁶, 3.0×10⁶, 4.0×10⁶, or 5.0×10⁶ total cells is effective.

Pharmaceutical compositions comprising the cells of the present disclosure (e.g., monocytes and/or stem cells) may be transplanted into a subject more than once. For example, the pharmaceutical composition comprising the cells of the present disclosure may be transplanted into a subject repeatedly until the condition of the subject improves.

Pharmaceutical compositions of the present disclosure, e.g., comprising cells and/or a therapeutic agent(s), may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, subcutaneous, intradermal, transdermal, transmucosal, intraosseous infusion (the process of injecting directly into the marrow of a bone), rectal, oral, nasal, transdermal, topical, or intramuscular administration.

For example, subjects of interest may be engrafted, infused, or transplanted with the cells of the present disclosure by various routes. Such routes include, but are not limited to, intravenous administration, subcutaneous administration, administration to a specific tissue (e.g., focal transplantation), injection into the bone marrow cavity, and the like. If by infusion, cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

The ratio of stem cells to monocytes can be 1:1, but can be adjusted as desired or otherwise appropriate (e.g., at least, about, or no more than 0.1:1, 0.5:1, 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment or transplantation of cells may be assessed by any of various methods, such as, but not limited to, cytokine levels, time of administration, increase in muscle cells and/or function at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, or 28 days can signal the time for assessing the desired effect. Any such metrics are variables that can be adjusted according to well-known parameters in order to determine the effect of the variable on a response to the pharmaceutical compositions comprising the cells of the present disclosure. In addition, the transplanted cells can be co-transplanted with other agents, such as cytokines, extracellular matrices, cell culture supports, and the like.

Pharmaceutical compositions comprising the cells of the present disclosure can also be administered before, concurrently with, or after, other therapeutic agent(s).

Pharmaceutical compositions comprising the cells of the present disclosure and/or a therapeutic agent(s) typically comprise a pharmaceutically acceptable carrier and/or diluent (see below). As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

Pharmaceutical compositions of the present disclosure are formulated to be compatible with its intended route of administration.

Solutions or suspensions (e.g., comprising cells and/or a therapeutic agent(s)) used for parenteral, intradermal, or subcutaneous application can include the following components: a sterile diluent (pharmaceutically acceptable diluent) such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions may be co-administered with enzyme inhibitors or in an appropriate carrier such as liposomes. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

Depending on the route of administration, the active compound can be coated in a material to protect the compound from the action of enzymes, acids and other natural conditions which may inactivate the compound. For example, for administration of agents by other than parenteral administration (esp. for oral administration), it may be desirable to coat the agent with, or co-administer the agent with, a material to prevent its inactivation.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds including, e.g., viral particles are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, inhibitory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

### Methods for Detecting Cytokines or Chemokines

The activity or level of a cytokine or chemokine can be detected and/or quantified by detecting or quantifying the expressed polypeptide. The polypeptide can be detected and quantified by any of a number of means well-known to those of skill in the art. Accordingly, any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired cytokine/chemokine standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In certain embodiments, a method for measuring the cytokine/chemokine levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies may be commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

In some embodiments, agents that specifically bind to a cytokine/chemokine other than antibodies are used, such as peptides. Peptides that specifically bind to a cytokine/chemokine is well known in the art (e.g., receptor fragment for the cytokine/chemokine), and can also be identified by any means known in the art. For example, specific peptide binders of a biomarker protein can be screened for using peptide phage display libraries.

### EXAMPLES

### Example 1: Materials and Methods

### Cell Lines, Reagents, and Antibodies

HEp2 cells were obtained from ATCC and were maintained in Dulbecco's Modified Eagle Medium (DMEM) supplemented with 10% FBS. MSCs were purchased from Clonetics and cultured with the basal medium provided by the manufacturer. DPSCs were isolated as described previously (Paranjpe et al. (2007) Free Radic Biol Med 43: 1394-1408) and they were cultured in complete DMEM supplemented with 10% FBS. Recombinant IL-2 was obtained from the NIH repository. The NK, CD16+ and CD16− monocyte purification kits were obtained from Stem Cell technologies (Stem Cell, Vancouver, Canada). The ELISA kits for IFN-γ, VEGF and IL-6 were purchased from R&D Systems (Minneapolis, MN). The TNF-α ELISA was reported previously (Jewett et al. (1997) J Immunol 159: 4815-4822). The multiplex cytokine array kit was purchased from R&D Systems. The Fluorescein Isothiocyanate (FITC) conjugated Annexin V/Propidium Iodide (PI) kit was purchased from Coulter Immunotech (Miami, FL).

### Purification of Nk Cells, Total Monocytes and Cd162 Monocytes

PBMCs from healthy donors were isolated as described before (Jewett and Bonavida (1996) J Immunol 156: 907-915). Briefly, peripheral blood lymphocytes were obtained after Ficoll-hypaque centrifugation and adherence to the plate for 1 hour. Purified NK cells were negatively selected by using an NK cell isolation kit. The purity of NK cell population was found to be greater than 90% based on flow cytometric analysis of anti-CD16 antibody stained cells. The levels of CD3+ T cell staining in purified population of NK cells remained low at 2.4%+/−1%, similar to that obtained by the non-specific staining using isotype control antibody throughout the experimental procedures. The adherent subpopulation of PBMCs was detached from the tissue culture plates and the total population of monocytes and those depleted of CD16+ subsets (CD16−) were purified using isolation kits obtained from Stem Cell Technologies (Vancouver, Canada).

Greater than 95% purity was achieved for each subset based on flow cytometric analysis of CD14 and CD16 antibody stained monocytes. Written informed consents approved by UCLA Institutional Review Board (IRB) were obtained from the blood donors and all the procedures were approved by the UCLA-IRB.

### ELISA and Multiplex Cytokine Array Kit

Single ELISAs were performed as described previously. Fluorokine MAP cytokine multiplex kits were purchased from R&D Systems (Minneapolis, MN) and the procedures were conducted as suggested by the manufacturer. To analyze and obtain the cytokine concentration, a standard curve was generated by either two or three fold dilution of recombinant cytokines provided by the manufacturer. Analysis was performed using the Star Station software.

### ⁵¹Cr Release Cytotoxicity Assay

The ⁵¹Cr release assay was performed as described previously. Briefly, MSCs or DPSCs were co-cultured with irradiated (10 Gy) subsets of monocytes for 24-48 hours before they were labeled with ⁵¹Cr for 1 hour, after which they were washed and added to NK samples. In several experiments monocytes were sorted out from the stem cell co-cultures before they were labeled with ⁵¹Cr and added to the NK cells. 100% removal of monocytes from the stem cells was achieved when the sorted samples were checked either by microscopy or by flow cytometric analysis of stained cells. After a 4 hour incubation period, the supernatants were harvested and counted for released radioactivity. The percentage of cytotoxicity was calculated as follows:

$$\% Cytotoxicity = \frac{Experimental cpm - Spontaneous cpm}{Total cpm - Spontaneous cpm}$$

Lytic unit 30/10⁶ is calculated by using the inverse of the number of effector cells needed to lyse 30% of tumor target cells×100.

### Surface and DNA Staining and Apoptosis Assay

Staining was performed by labeling the cells with antibodies or propidium iodide and Annexin V as described previously.

### Luciferase Reporter Assay

Transfections were performed using NFkB Luciferase reporter vector and Lipofectamine 2000 reagent (Invitrogen, CA) in Opti-MEM media (Invitrogen, CA) for 18 hours after which they were adhered to the plate overnight before different immune effectors at 1:1 Effector to target ratios were added. The cells were then lysed with lysis buffer and the relative Luciferase activity was measured using the Luciferase assay reagent kit obtained from Promega (Madison, WI).

### Statistical Analysis

An unpaired, two-tailed student t-test was performed for the statistical analysis. One way ANOVA with a Bonferroni post test was used to compare the different groups.

### Example 2: Combining Autologous or Allogeneic Total Monocytes and CD16+ Monocytes to Protect and Increase the Expansion and Function of Stem Cells

MSCs were Selected Based on their Phenotypic and Functional Properties.

MSCs were CD166+CD105+CD99+CD34−CD45− and CD14− based on the flow cytometric analysis (data not shown). In addition, MSCs were capable of differentiating to osteogenic, chondrogenic and adipogenic lineages (data not shown, www.clonetics.com).

### IL-2 Activated NK Cells are Potent Inducers of Cell Death in MSCs

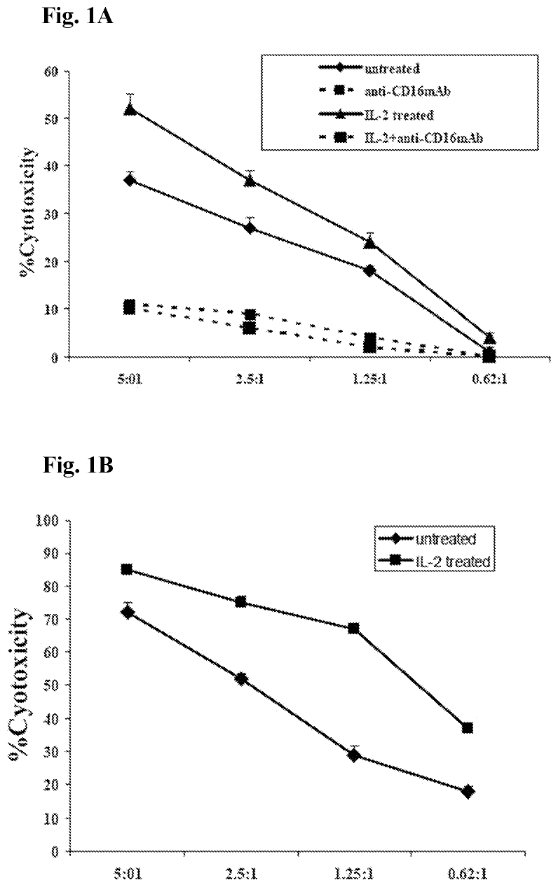

Highly purified human NK cells were cultured with and without IL-2 for 8-12 hours before they were added to ⁵¹Cr labeled MSCs (FIG. 1A). Addition of non activated NK cells had lower cytotoxic activity against both MSCs (FIG. 1A) and K562s (FIG. 1). However, activation with IL-2 increased cytotoxicity and resulted in a significant lysis of these cells (p<0.05)(FIG. 1).

### Anti-CD16 Antibody Induces Death of Untreated and IL-2 Treated NK Cells and Inhibits NK Cell Mediated Lysis of MSCs

NK cells were left untreated or treated with anti-CD16 antibody and/or IL-2 for 8-12 hours before they were added to ⁵¹Cr labeled MSCs. As shown in a number of studies, anti-CD16 mAb treatment induced death of the NK cells and inhibited NK cell cytotoxicity against MSCs (p<0.05) (FIG. 1). Addition of the combination of IL-2 and anti-CD16 treatment also induced NK cell death and inhibited NK cell cytotoxicity against MSCs when compared to IL-2 activated NK cells (p<0.05) (FIG. 1A).

### Monocytes Prevent NK Cell Induced Death of MSCs

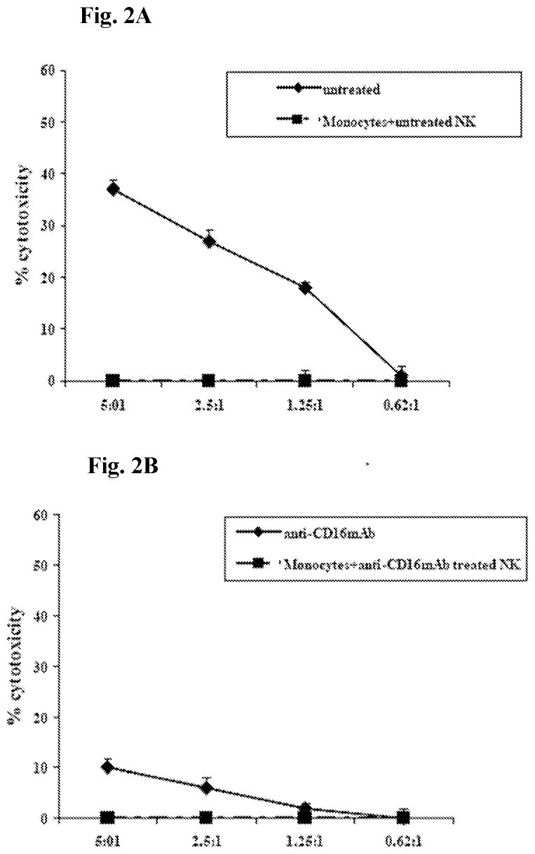

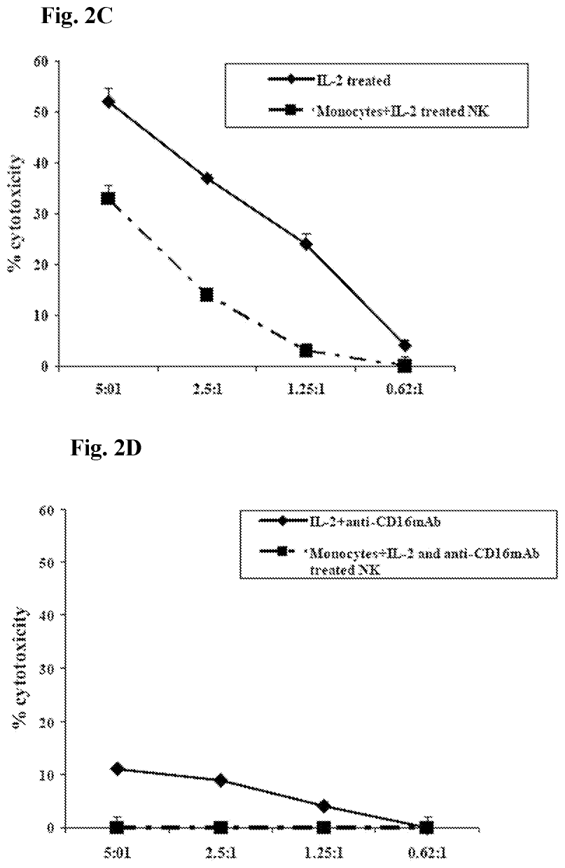

Autologous monocytes were purified from PBMCs and irradiated as indicated. MSCs were cocultured with allogeneic irradiated monocytes for 24-48 hours before they were labeled with ⁵¹Cr and used in the cytotoxicity assays against NK cells. NK cells were left untreated (FIG. 2A) or pretreated with anti-CD16 antibody (FIG. 2B) or IL-2 (FIG. 2C) or combination of IL-2 and anti CD16mAb (FIG. 2D) for 24-48 hours before they were used in the cytotoxicity assays against MSCs. Addition of monocytes to MSCs significantly protected the MSCs against NK cell mediated cytotoxicity (p<0.05) (FIG. 2). Significant inhibition of NK cell cytotoxicity by monocytes could be observed against all treated NK samples (p<0.05) (FIG. 2). Monocytes also increased the levels of Alkaline Phosphatase staining in MSCs and prevented decrease in Alkaline Phosphatase expression induced by IL-2 activated NK cells. Either untreated or anti-CD16 antibody treated live or irradiated Monocytes did not mediate any cytotoxicity against MSCs or K562 cells. Overall, these experiments indicated that monocytes protect MSCs against NK cell mediated lysis.

Monocytes Increase the Levels of IL-6, TNF-α and VEGF when Cultured with MSCs.

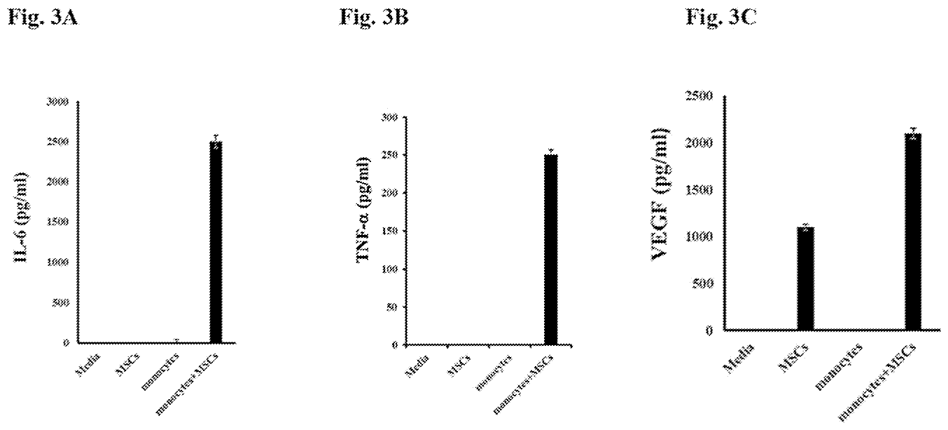

Irradiated Monocytes were added to MSCs for 24-48 hours before the supernatants were removed, and the levels of IFN-γ (0.0 μg/ml, data not shown) IL-6 (FIG. 3A), TNF-α (FIG. 3B) and VEGF (FIG. 3C) were determined using multiplex cytokine array assay. Monocytes increased the levels of IL-6 and TNF-α and VEGF in co-cultures with MSCs (p<0.05) (FIG. 3). Secretion of VEGF was only observed in samples containing MSCs, and monocytes further increased secreted levels of VEGF by MSCs (p<0.05) (FIG. 3C). These results indicated that monocytes increased all of the above-mentioned cytokines in co-cultures with MSCs.

### Monocytes Raise the Overall Survival and Function of MSCs

Monocytes prevented death of MSCs as assessed in ⁵¹Cr release assay and as seen with alkaline phosphatase staining of MSCs. Therefore, monocytes raise the survival of MSCs. Thus, increased survival of MSCs by monocytes increases the overall function of MSCs resulting in a synergistic increase in IL-6, TNF-α, and VEGF (FIG. 3). The increase in survival of MSCs by monocytes is also evident when assessed morphologically.

### Monocytes are Major Inducers of NFkB in Cells.

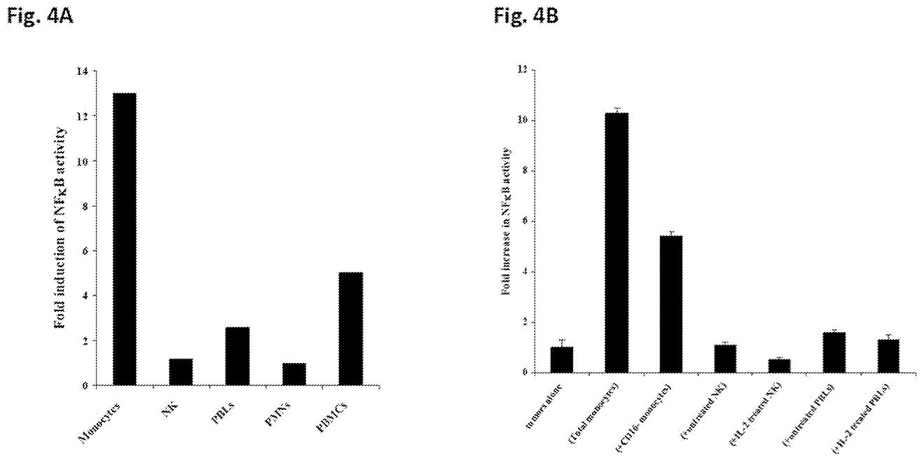

Purified subpopulations of the immune effectors, namely, highly purified monocytes, Natural Killer cells, PMNs and total populations of peripheral blood mononuclear cells and enriched peripheral blood lymphocytes were co-cultured with NFkB reporter transfected 293T and HEp2 cells (FIG. 4A), and the levels of NFkB activity were determined after an overnight incubation. As shown in FIG. 4A monocytes induced the highest levels of NFkB whereas all the other populations including NK cells had minimal effect (p<0.05).

### CD16+ as Well as CD16− Subsets of Monocytes are Potent Inducers of NFkB and Prevent NK Cell Mediated Cytotoxicity Against MSCs.

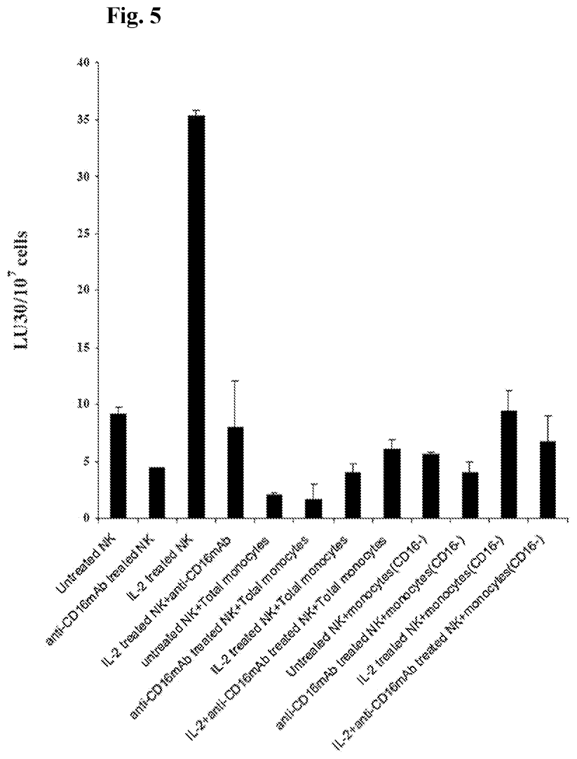

To determine if all or a subset of monocytes were responsible for the prevention of NK cell mediated cytotoxicity against MSCs we depleted CD16+ subpopulation of monocytes from the total monocyte population (CD16−) and compared it to the activity of monocytes containing both the CD16+ and CD16− subpopulations. Depletion of CD16+ subset of monocytes from the total population of monocytes decreased approximately 40%-50% of NFkB activity in HEp2 cells (FIG. 4B). Furthermore, although CD16− subset of monocytes could still prevent IL-2 treated NK cell mediated cytotoxicity against MSCs significantly (p<0.05) (9.4 LU vs. 35.2 LU), when compared to total population containing CD16+ subpopulation, they had lower ability to prevent NK cell mediated cytotoxicity (9.4 LU vs. 4 LU) (FIG. 5). There was an 8.8 fold decrease in the levels of IL-2 activated NK cell cytotoxicity when total monocytes were added to the MSCs whereas only 3.74 fold decrease in NK cell cytotoxicity could be seen when CD16-subset of monocytes were added to the MSC cultures (FIG. 5). This represents an approximately 57% decrease in the ability of CD16− monocytes to prevent NK cell mediated cytotoxicity against MSCs when compared to total population of monocytes (FIG. 5). Considering that CD16+ subset of monocytes comprise only a small fraction of the total population of monocytes, they contribute significantly to the survival and function of both NK cells as well as the MSCs.

Based on these results we will combine stem cells such as MSCs or DPSCs or ESCs or iPSCS with irradiated monocytes before transplantation in the patients, e.g., in the weakened muscle, to increase their survival and function to not only guard against CD8+ T cell activation in diseases such as ALS but also repair the lost function in muscles and CNS. Indeed, this strategy can be used in implantation of any stem cells to provide survival to the stem cells.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

What is claimed is:

1. A pharmaceutical composition comprising irradiated monocytes and stem cells.

2. The pharmaceutical composition of claim 1, wherein the stem cells are preconditioned to enhance the survival.

3. The pharmaceutical composition of claim 1 or 2, wherein the stem cells are preconditioned with exposure to hypoxia (e.g., 0.5% oxygen), hyperoxia (100% oxygen), and/or sevoflurane.

4. The pharmaceutical composition of any one of claims 1-3, further comprising at least one additional agent that enhances the survival of the stem cells.

5. The pharmaceutical composition of claim 4, wherein the at least one additional agent is selected from TGF-α, Z-VAD-FMK pan-caspase inhibitor, simvastatin, rosuvastatin, an inhibitor of Janus kinase (JAK), and an inhibitor of p38.

6. The pharmaceutical composition of any one of claims 1-5, wherein the pharmaceutical composition comprises a biomaterial, optionally wherein the biomaterial comprises hydrogel (e.g., thermosensitive hydrogel) and/or a cell sheet.

7. The pharmaceutical composition of any one of claims 1-6, wherein the stem cells are selected from mesenchymal stem cells (MSCs), embryonic stem cells (ESCs), dental pulp stem cells (DPSCs), hematopoietic stem cells (HSCs), stem cells from human exfoliated deciduous teeth (SHED), and induced pluripotent stem cell (iPSCs).

8. The pharmaceutical composition of any one of claims 1-7, wherein the monocytes are gamma-irradiated.

9. The pharmaceutical composition of any one of claims 1-8, wherein the composition comprises monocytes that express CD16 on the cell surface.

10. The pharmaceutical composition of any one of claims 1-9, wherein at least 50%, 60%, 70%, 80%, 90%, or 95% of the monocytes in the composition express CD16 on the cell surface.

11. The pharmaceutical composition of any one of claims 1-10, wherein the composition produces or secretes at least one cytokine or growth factor.

12. The pharmaceutical composition of claim 11, wherein the at least one cytokine or growth factor is selected from IL-6, TNF-α, and VEGF.

13. The pharmaceutical composition of claim 11 or 12, wherein the at least one cytokine or growth factor comprises IL-6, TNF-α, and VEGF.

14. The pharmaceutical composition of any one of claims 1-13, wherein the monocytes increase the activity of NFkB in the stem cells.

15. The pharmaceutical composition of any one of claims 1-14, wherein the monocytes (i) inhibit the NK cell-mediated death of the stem cells, and/or (ii) enhance the survival of the stem cells.

16. A method of treating a subject in need of stem cell transplantation, the method comprising transplanting the composition of any one of claims 1-15 into the subject.

17. The method of claim 16, wherein the subject is afflicted with a neurodegenerative disease, paralysis, cancer (e.g., leukemia, lymphoma, multiple myeloma), liver cirrhosis, or ischemic heart disease.

18. The method of claim 16 or 17, wherein a peripheral nerve function is decreased in the subject.

19. The method of any one of claims 16-18, wherein the subject is afflicted with a traumatic nerve injury, muscle atrophy, muscular denervation atrophy, neuropathy, or motor neuron disease.

20. The method of any one of claims 16-19, wherein the subject is afflicted with a peripheral nerve injury, amyotrophic lateral sclerosis (ALS), spinal muscular atrophy, spinocerebellar ataxia, post-polio syndrome, and Charcot-Marie-Tooth disease.

21. The method of claim 16 or 17, wherein a central nerve function is decreased in the subject.

22. The method of claim 16, 17, or 21, wherein the subject is afflicted with Alzheimer's disease, Parkinson's disease, or Huntington's disease.

23. A method of promoting neuromuscular regeneration in a subject, the method comprising transplanting the composition of any one of claims 1-15 into the subject.

24. The method of claim 23, wherein the subject is afflicted with a neurodegenerative disease.

25. The method of claim 23 or 24, wherein a peripheral nerve function is decreased in the subject.

26. The method of any one of claims 23-25, wherein the subject is afflicted with a traumatic nerve injury, muscle atrophy, muscular denervation atrophy, neuropathy, or motor neuron disease.

27. The method of any one of claims 23-26, wherein the subject is afflicted with a peripheral nerve injury, amyotrophic lateral sclerosis (ALS), spinal muscular atrophy, post-polio syndrome, spinocerebellar ataxia, or Charcot-Marie-Tooth disease.

28. The method of claim 23 or 24, wherein a central nerve function is decreased in the subject.

29. The method of claim 23, 24, or 28, wherein the subject is afflicted with Alzheimer's disease, Parkinson's disease, or Huntington's disease.

30. The method of any one of claims 16-29, wherein the monocytes are autologous or allogeneic to the subject.

31. The method of any one of claims 16-30, wherein the stem cells are autologous or allogeneic to the subject.

32. The method of any one of claims 16-31, further comprising administering to the subject at least one additional agent that enhances the survival of the stem cells.

33. The method of claim 32, wherein the at least one additional agent is administered before, after, or concurrently with the composition of anyone of claims 1-15.

34. The method of claim 32 or 33, wherein the at least one additional agent is selected from TGF-α, Z-VAD-FMK pan-caspase inhibitor, simvastatin, rosuvastatin, an inhibitor of Janus kinase (JAK), and an inhibitor of p38.

35. The method of any one of claims 16-34, wherein the composition is transplanted intramuscularly or by intraosseus infusion.

36. The method of any one of claims 16-35, wherein the subject is a mammal.

37. The method of any one of claims 16-36, wherein the subject is a human.

38. A method of enhancing the survival of stem cells, the method comprising contacting the stem cells with monocytes.

39. The method of claim 38, wherein the stem cells are contacted with monocytes in vitro, ex vivo, or in vivo.

40. The method of claim 38 or 39, wherein the monocytes express CD16 on the cell surface.

41. The method of any one of claims 38-40, wherein at least 50%, 60%, 70%, 80%, 90%, or 95% of the monocytes, which contact the stem cells, express CD16 on the cell surface.

42. The method of any one of claims 38-41, wherein the monocytes are irradiated.

43. The method of any one of claims 38-42, wherein the monocytes are gamma-irradiated.

44. The method of any one of claims 38-43, wherein the stem cells are selected from mesenchymal stem cells (MSCs), embryonic stem cells (ESCs), dental pulp stem cells (DPSCs), hematopoietic stem cells (HSCs), stem cells from human exfoliated deciduous teeth (SHED), and induced pluripotent stem cell (iPSCs).

45. The method of any one of claims 38-44, wherein the monocytes produce or secrete at least one cytokine or growth factor in the presence of the stem cells.

46. The method of claim 45, wherein the at least one cytokine or growth factor is selected from IL-6, TNF-α, and VEGF.

47. The method of claim 45 or 46, wherein the at least one cytokine or growth factor comprises IL-6, TNF-α, and VEGF.

48. The method of any one of claims 38-47, wherein the monocytes increase the activity of NFkB in the stem cells.

49. The method of any one of claims 38-48, wherein the monocytes (i) inhibit the NK cell-mediated death of the stem cells, and/or (ii) enhance the survival of the stem cells.

50. The method of any one of claims 38-49, further comprising contacting the stem cells with at least one additional agent that enhances the survival of the stem cells.

51. The method of claim 50, wherein the at least one additional agent is selected from TGF-α, Z-VAD-FMK pan-caspase inhibitor, simvastatin, rosuvastatin, an inhibitor of Janus kinase (JAK), and an inhibitor of p38.

52. The method of any one of claims 38-51, wherein the stem cells are preconditioned to enhance survival.

53. The method of any one of claims 38-52, wherein the stem cells are preconditioned with exposure to hypoxia (e.g., 0.5% oxygen), hyperoxia (100% oxygen), or sevoflurane.
